(19) United States
(12) Patent Application Publication
(10) Pub. No.: US 2018/0020981 A1
(43) Pub. Date: Jan. 25, 2018

(54) MODULAR BIOFLUID SENSING SUBSYSTEMS AND DEVICES

(71) Applicant: Eccrine Systems, Inc., Cincinnati, OH (US)

(72) Inventors: Jason Heikenfeld, Cincinnati, OH (US); Austin Morgan, Cincinnati, OH (US); Jacob A. Bertrand, Norwood, OH (US)

(73) Assignee: Eccrine Systems, Inc., Cincinnati, OH (US)

(21) Appl. No.: 15/655,794

(22) Filed: Jul. 20, 2017

Related U.S. Application Data
(60) Provisional application No. 62/364,939, filed on Jul. 21, 2016.

(51) Int. Cl. A61B 5/00; A61B 5/01; A61B 5/145; A61B 5/053

(52) U.S. Cl. CPC A61B 5/6832; A61B 5/0533; A61B 5/4266; A61B 5/01; A61B 5/14539

(57) ABSTRACT

The disclosed invention provides a modular biofluid sensing device configured to be worn on an individual's skin. The device includes at least one primary module, at least one sensing module, and at least one specialized module. The various subsystems, components, and materials making up a biofluid sensing device are arranged for modular distribution and assembly according to a number of different organizational criteria. These criteria include distributing components into modules based on the requirements of a biofluid sensing device application, manufacturing considerations, component cost, and component lifespan.

### CROSS-REFERENCE TO RELATED APPLICATIONS

This application relates to U.S. Provisional No. 62/364,939, filed Jul. 21, 2016, the disclosure of which is hereby incorporated by reference herein in its entirety.

### STATEMENT REGARDING FEDERALLY SPONSORED RESEARCH OR DEVELOPMENT

The present invention was made outside any support from the U.S. Government.

### BACKGROUND OF THE INVENTION

Despite the many ergonomic advantages of eccrine perspiration (sweat) compared to other possible biofluids (particularly in “wearable” devices), sweat remains an underrepresented source of biomarker analytes compared to blood, urine, and saliva. Upon closer comparison to other non-invasive biofluids, the advantages may even extend beyond ergonomics: sweat might provide superior analyte information. A number of challenges, however, have historically kept sweat from assuming a more prominent place among clinical sampling modalities. These challenges include very low sample volumes (nL to μL), unknown concentration due to evaporation, filtration and dilution of large analytes, mixing of old and new sweat, and the potential for contamination from the skin surface. More recently, rapid progress in wearable sweat sampling and sensing devices has resolved several of these historical challenges. However, this recent progress has also been limited to high concentration analytes (μM to mM) sampled at high sweat rates (>1 nL/min/gland, e.g. athletic applications). Progress will become much more challenging as sweat biosensing moves towards use with sedentary users (low sweat rates or not sweating at all) and/or towards low concentration analytes (pM to nM). Furthermore, the solutions to resolving these problems will be highly multidisciplinary, and may require source components that will be very dissimilar in their manufacturing infrastructure or cost profile. As a result, monolithic integration of all materials and components in a sweat sensing device, in many circumstances may be impractical, or render certain applications prohibitively expensive. Furthermore, some materials and components may be needed for nearly all sweat sensing applications, whereas other materials and components would be needed only for niche applications. In such cases, modular techniques are required to allow efficient integration of broadly applicable materials, or components with niche application materials or components. Additionally, modular techniques may allow the distribution of expensive components among disposable and reusable modules, or can allow one-use or limited-use components to be efficiently combined with components capable of longer lifespan.

Many of the drawbacks and limitations currently facing sweat sensing and other biofluid sensing modalities can be resolved by creating novel and advanced interplays of chemicals, materials, sensors, electronics, microfluidics, algorithms, computing, software, systems, and other features or designs, in a manner that affordably, effectively, conveniently, intelligently, or reliably brings biofluid to sensors and to biofluid preparing or concentrating subsystems. By doing do, sweat sensing could become a much more compelling paradigm as a biosensing platform, and other biofluid sensing modalities can be improved.

### SUMMARY OF THE INVENTION

The disclosed invention provides a wearable biofluid sensing device configured for the modular distribution and assembly of a variety of subsystems, components, and materials. These include the modular distribution of components based on biofluid sensing device application, manufacturing considerations, cost considerations, and component lifespan.

### BRIEF DESCRIPTION OF THE DRAWINGS

The objects and advantages of the present invention will be further appreciated in light of the following detailed descriptions and drawings in which:

FIG. 1 is a cross-sectional view of a prior art device lacking modular component distribution and assembly.

FIG. 2A-2C are cross-sectional views of embodiments of the disclosed invention with multiple modular subsystems.

FIG. 3A is a cross-sectional view of an embodiment of the disclosed invention with greater detail shown for modules 210 and 220 of FIGS. 2A-2C.

FIG. 3B is a cross-sectional view of an embodiment of the disclosed invention with greater detail shown for module 230 of FIGS. 2A-2C.

FIG. 3C is a cross-sectional view of an embodiment of the disclosed invention with greater detail shown for module 230 of FIGS. 2A-2C.

FIG. 3D is a cross-sectional view of an embodiment of the disclosed invention with greater detail shown for module 210 of FIGS. 2A-2C.

FIG. 4 is a cross-sectional view of an embodiment of the disclosed invention with a modular assembly of subsystems, components, and materials.

### DEFINITIONS

Before continuing with the background, a variety of definitions should be made, these definitions gaining further appreciation and scope in the detailed description and embodiments of the present disclosure.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biosensor” means any type of sensor that measures a state, presence, flow rate, solute concentration, solute presence, in absolute, relative, trending, or other ways in a biofluid. Biosensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a fluid sensing device.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

“Chronological assurance” means the sampling rate or sampling interval that assures measurement(s) of analytes in sample in terms of the rate at which measurements can be made of new fluid analytes as they enter the sample. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

As used herein, “continuous monitoring” means the capability of a device to provide at least one measurement of sweat determined by a continuous or multiple collection and sensing of that measurement or to provide a plurality of measurements of sweat over time.

“Biofluid sensor data” means all the information collected by fluid sensing device sensor(s) and communicated to a user or a data aggregation location.

“Correlated aggregated fluid sensor data” means fluid sensor data that has been collected in a data aggregation location and correlated with outside information such as time, temperature, weather, location, user profile, other biofluid sensor data, or any other relevant data.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Sweat volume” is the fluidic volume in a space that can be defined multiple ways. Sweat volume may be the volume that exists between a sensor and the point of generation of sweat or a solute moving into or out of sweat from the body or from other sources. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

“Microfluidic components” are channels in polymer, textiles, paper, or other components known in the art of microfluidics for guiding movement of a fluid or at least partial containment of a fluid.

“Sweat sampling rate” is the effective rate at which new sweat or sweat solutes, originating from the sweat gland or from skin or tissue, reaches a sensor which measures a property of sweat or its solutes. Sweat sampling rate, in some cases, can be far more complex than just sweat generation rate.

“Sweat stimulation” is the direct or indirect causing of sweat generation by any external stimulus, the external stimulus being applied for the purpose of stimulating sweat. One example of sweat stimulation is the administration of a sweat stimulant such as pilocarpine. Going for a jog, which stimulates sweat, is only sweat stimulation if the subject jogging is jogging for the purpose of stimulating sweat.

As used herein, “microfluidic components” are channels in polymer, textiles, paper, or other components known in the art of microfluidics for guiding movement of a fluid or at least partial containment of a fluid.

As used herein, “advective transport” is a transport mechanism of a substance or conserved property by a fluid due to the fluid's bulk motion.

“Diffusion” means the net movement of a substance from a region of high concentration to a region of low concentration. This is also referred to as the movement of a substance down a concentration gradient.

A “module” is a component or components which are fabricated individually and integrated with at least one other modeling during assembly of a sweat sensing device.

“Volume reducing component” means any component which reduces the sweat volume as taught in PCT/US15/32893, which is hereby incorporated by reference herein in its entirety.

“Volume reducing wicking component” means any component as taught in PCT/US16/43771, which is hereby incorporated by reference herein in its entirety.

“Sweat stimulating component” means any component as taught in PCT/US14/61083, PCT/US16/17726, U.S. Ser. No. 15/186,925, and PCT/US16/50928, which are hereby incorporated by reference herein in their entirety.

“Electroporation component” means any component as taught in PCT/US17/13453, which is hereby incorporated by reference herein in its entirety.

“Sensor component” means any component or components which measure a solute in sweat, a property of sweat, or the presence of sweat. Sensors can be thermal, flow, impedance, potentiometric, ion-selective, amperometric, enzymatic, aptamer, antibody, fluorescent, colorimetric, surface-plasmon resonance, acoustic, resonant, MEMs, or any other sensor suitable for sensing sweat in at least one measurement.

“Flow rate sensor” means any component or components which measure the flow rate of sweat or other biofluid in at least one portion of a biofluid sensing device.

“Primary module” means any component or components that may contain “sensor components”, “stimulating components”, “volume reducing components”, and/or “volume reducing wicking components”.

“Sensing module” means any component or components fabricated separately from the primary module and specialized module, that provides one or more generally applicable sensors, such as one or more ion-selective electrodes, a biofluid flow rate sensor, a pH sensor, a temperature sensor, a galvanic skin response sensor, or a skin impedance sensor.

“Specialized module” means any component or components, fabricated separately from the primary module and sensing module, that provides a specialized and application-specific purpose in a biofluid sensing device, such as one or more electrochemical aptamer-based sensors, ion-selective electrode sensors; amperometric sensor, potentiometric sensor, enzymatic sensor, antibody sensor, optical sensor, surface-plasmon sensor, acoustic sensor, resonant sensor, micro-electro-mechanical MEMs sensor, biofluid sample concentration component, osmotic pump component, wicking component, or a sample collection and storage component, including those as taught in PCT/US16/58356, and PCT/US17/23399 which are hereby incorporated by reference herein in their entirety.

### DETAILED DESCRIPTION OF THE INVENTION

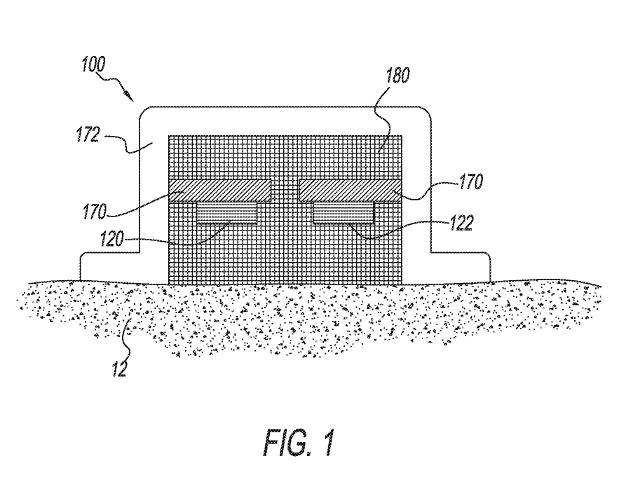

With reference to FIG. 1, a prior art device on skin 12 contains a skin-adhesive polymer seal 172, such as medical adhesive on PET film; at least one sensor 120, 122; at least one substrate 170, such as a PET film; and at least one wicking material 180, such as a hydrogel. The entire device is fabricated monolithically, by adding basic materials step-by-step. For instance, sensors 120, 122 could be ion-selective electrodes deposited by screen printing on PET, attached to substrate 170, surrounded with hydrogel 180, and then encapsulated by seal 172.

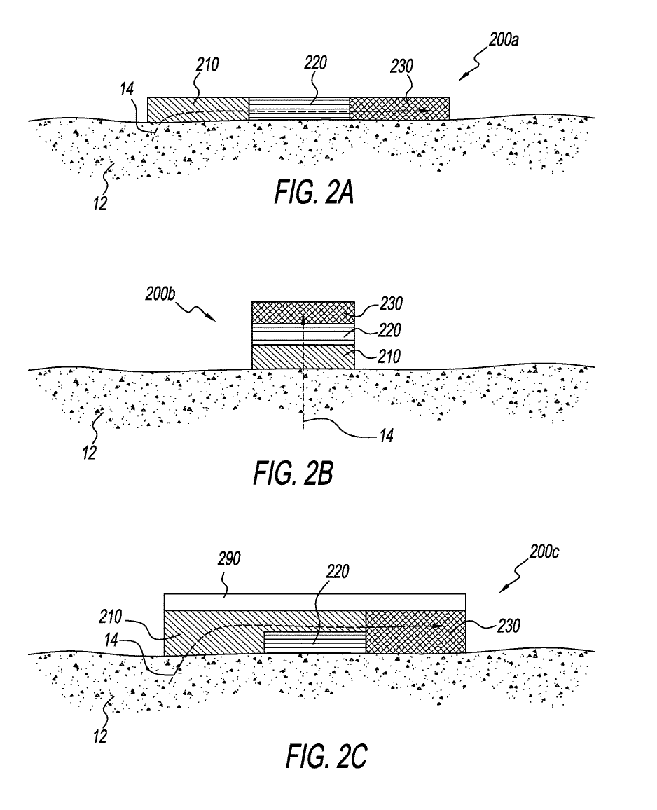

With reference to FIG. 2A, a modular biofluid sensing device 200a contains a primary module 210 which comprises at least one of the following: a sweat stimulating component; a thermal flow measurement sensor; a volume reducing component; a wicking volume reducing component; and an electroporation component. FIG. 2A further contains at least one sensing module 220, and at least one specialized module 230. Each of the modules 210, 220, 230, is functionally connected as needed and integrated with the others into a single device. Sweat or other biofluid is transported in a manner that is dominantly horizontal along skin 12, from skin in the direction of the arrow 14 through the primary module 210, through sensing module 220, and to specialized module 230.

With reference to FIG. 2B, a biofluid sensing device 200b with an alternate arrangement of the modules of FIG. 2A is shown, where sweat is transported in a manner that is dominantly away from skin 12, in the direction of the arrow 14 through the primary module 210, through sensing module 220, and to specialized module 230.

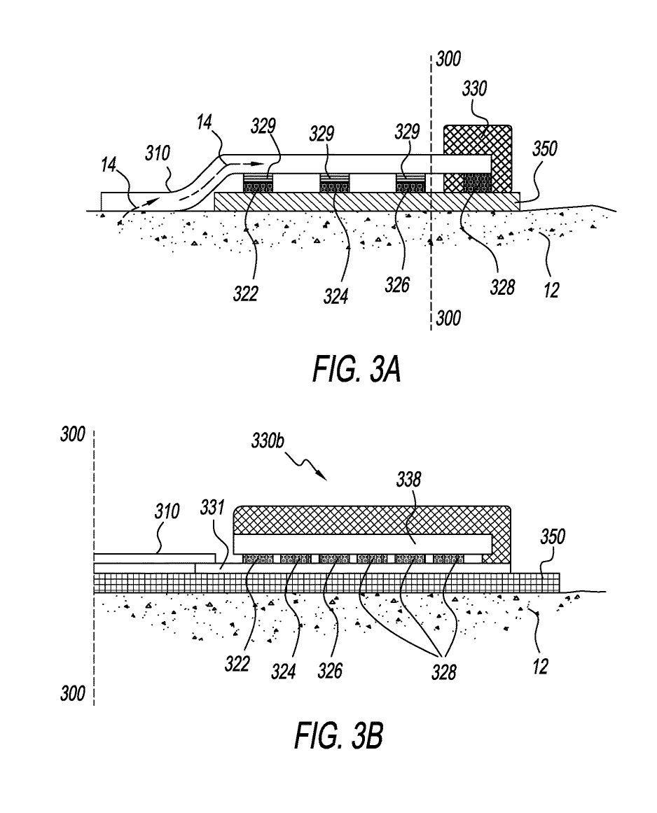

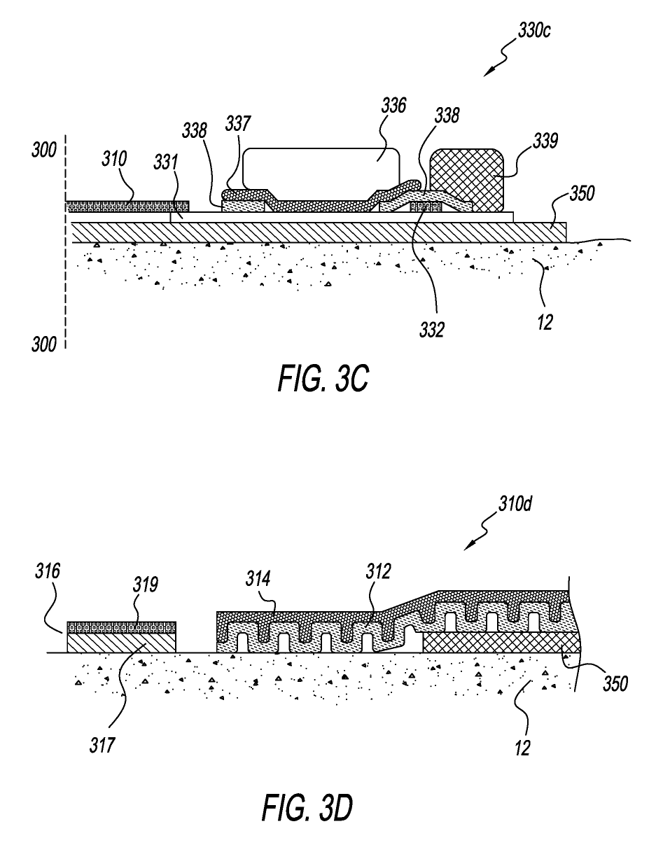

With reference to FIG. 2C, a device 200c with an alternate arrangement of the modules of FIGS. 2A and 2B are shown, including modular electronics 290, which can be integrated using one or more techniques, including those taught in PCT/US15/32843. A primary module 210 carries sweat from skin 12 in the direction of the arrow 14 into contact with and over a sensing module 220 which contains sweat sensors, e.g., ion selective electrode sensors or a pH sensor, and to a specialized module 230. A more detailed description of this example embodiment will be provided in FIG. 3A.

With reference to FIG. 3A, a biofluid sensing device includes a wicking volume reducing component as a primary module 310 and at least one specialized module 330 (one is shown) that receives sweat from the primary module 310. Specialized module 330 could be simply a wicking hydrogel, but could also include specialized sensors 328, or be otherwise more sophisticated, as will be described for FIG. 3B. The device includes a sensing module 320, which is composed of at least one biofluid sensor 322, 324, 326, and a substrate 350. In an exemplary embodiment, the sensing module 320 includes at least one sensor for Na⁺ or Cl⁻, at least one sensor for K⁺, at least one pH sensor, and at least sweat rate sensor, e.g., a thermal flow rate sensor from Sensiron Corporation, a volumetric sweat rate sensor, or other suitable biofluid flow rate sensor. This suite of sensors chosen for the sensing module is intended to provide key information for at least one specialized sensor 328, e.g., an electrochemical aptamer-based sensor for cortisol, located in the specialized module 330. The interpretation of sweat measurements taken by the specialized sensor 328 could be affected by changes in sweat sample pH or salinity, and the sensor's chronological accuracy is dependent on sweat flow rate to the sensor.

To illustrate the modular nature of component distribution and assembly, the primary module 310 is, e.g., a disposable microfluidic wicking component, that interfaces with the reusable sensing module 320 and its sensors 322, 324, 326, by pressing the primary module against the sensing module, and securing the modules together by means of a simple mechanical interaction, such as an adhesive or click attachment means. Interfacing the primary module 310 with the sensing module 320 thus puts the wicking component in fluid communication with the sensors 322, 324, 326. An optional hydrogel or other wicking material 329 can be placed between at least one of the sensors 322, 324, 326 and wick 310, to improve the transfer of biofluid sample or biofluid analytes from the wick 310 to the sensors 322, 324, 326. Additionally, a reusable specialized module 330 is connected to the primary module 310 by means similar to the connection of the primary module to the sensing module, e.g., by simple physical contact or a mechanical interaction, so that the wick is in fluid communication with the specialized module. In some embodiments, the device includes a vapor barrier layer (not shown) over primary module 310, which prevents or reduces biofluid sample evaporation out of the device. In other embodiments, a vapor barrier layer (not shown) could be located above the substrate 350 and below the sensors 322, 324, 326 to prevent vapor from escaping once it has entered the device. Alternatively, the device may have both such vapor barrier layers, which may be separate component(s) or ray be manufactured/integrated with the substrate 350, the primary module 310, or another module as necessary. Alternatively, the sensing module 320 could have its own microfluidic component that is placed in fluidic communication with both the primary module and the specialized module (not shown).

With reference to FIG. 3B, which represents the portion of the device of FIG. 3A that appears to the right of the line 300, an example specialized module 330b is illustrated in greater detail. A portion of the primary module 310, e.g., a wicking or volume reducing component, and a substrate 350 are shown for reference. A wicking or microfluidic component 314 brings a sweat sample to at least one sensor 332, 334, 336, and a sensor suite 338 which includes for, example, three sensors of the same sensor type, e.g., three EAB sensors for detecting sweat cortisol, or three amperometric sensors for lactate or glucose. The specialized module sensors 332, 334, 336 may also include a pH sensor, a salinity sensor, a sweat flow rate sensor, or a temperature sensor. These sensors would, for example, provide additional data relative to sweat sample pH or temperature near the sensor suite 338, which may experience different conditions than near the skin 12, or upstream within the device. The specialized module 330b also includes a wicking component 339, e.g., a hydrogel, and a substrate 338, upon which the sensors 332, 334, 336, 338 are fabricated and mounted. The wicking or microfluidic component 314 could be fabricated along with the substrate 338, gel 339, and the sensors 332, 334, 336, 338, e.g., the specialized module components are fabricated together as one complete and reusable module and interfaced with a disposable primary module and a limited-reuse sensing module.

With reference to FIG. 3C, which represents the portion of the device of FIG. 3A that appears to the right of the line 300, an alternate embodiment of a specialized module 330c includes a biofluid sample concentration component and at least one sensor 332. A wicking material 339 is also provided, as is a wicking or microfluidic component 331. The module 330c is fabricated upon a substrate 338. The biofluid (in this case sweat) sample concentration component includes an osmotic pumping material 336, which could be a large organic salt or sugar, or a strongly wicking material, such as a hydrogel. Sweat sample concentration also includes a selectively permeable membrane 337, e.g., a forward osmosis membrane, that is in fluidic communication with the wicking component 331. The sensor 332 could be a μM EAB sensor for cortisol. The concentration component achieves, for example, a 10× to 1000× concentration of cortisol relative to the original sweat cortisol concentration prior to the sweat sample entering the concentration component.

With reference to FIG. 3D, an embodiment of a primary module 310d depicted in additional detail. the primary module 310d includes a hydrophilic gold electroporation electrode 312 that is interfaced with a geometric channel 314, constructed of, e.g., a polymer. The hydrophilic properties of the electrode 312 allow the geometries of the channel 314 both to wick sweat and to act as a wicking volume reducing component. The primary module also includes a sweat stimulation component 316 comprised of sweat stimulant gel 317 and iontophoresis electrode 319. Sweat stimulation and collection, in this example, may be accomplished via sudo-motor axon reflex sweating.

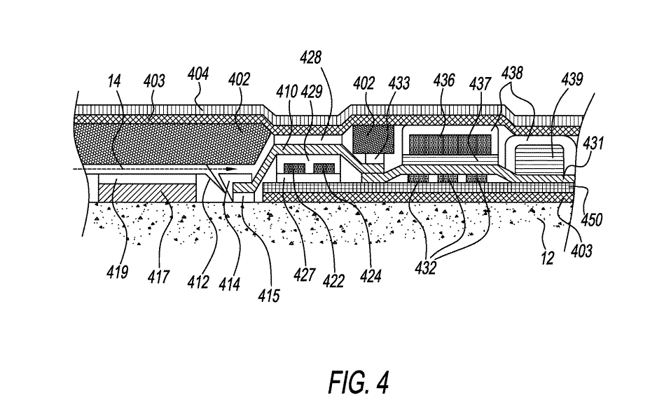

With reference to FIG. 4, a partial view of a fully detailed embodiment of the disclosed invention is provided. The modular device contains a primary module, a sensing module, and a specialized module, as well as the following: a filler material constructed of sponge or memory foam 402; an adhesive 403, e.g., an acrylate or medical adhesive; a textile covering 404; and a substrate 450. The primary module includes the following: a wicking volume reducing component 410; an iontophoresis electrode 419; a <1 mm thick sweat stimulant gel comprising a carbachol sweat stimulant and agar 417; a first rigid molded polymer 412; a second rigid molded polymer 414 designed to interact with the first molded polymer 412; and an electroporation electrode 415. The sensing module includes the following: at least one sensor, reference electrode or counter electrode 422, 424; a memory foam or other self-leveling material 427; a hydrogel spacer 429 for enhancing fluidic, adventive or diffusive contact between the substrate 450 and a rigid polymer or metal component 428; where the spacer 429 further provides a clamping pressure between the rigid component 428 and the substrate 450 such that the wicking volume reducing component 410 and the sensors 422, 424 are in fluidic communication at all times. The specialized module includes the following: a wicking or microfluidic component 431; at suite of three EAB sensors for vasopressin 432, where the EAB sensors have a linear range of detection centered around 100 nM; an osmosis pumping material 436; a forward osmosis membrane 437 with a molecular weight cutoff of approximately 100 to 200 Da; a polymer seal 438; and a wicking pumping material 439.

With further reference to FIG. 4, the modular device operates as follows: the first rigid molded polymer 412 is mechanically actuated in the direction of the arrow 14 so that the first polymer 412 interacts with and lifts up the second molded polymer 414, so that the iontophoresis electrode 419, and sweat stimulant gel 417 are moved underneath the electroporation electrode 414 to provide iontophoretic sweat stimulation every 2 to 12 hours, or as needed. The first molded polymer 412 can then be retracted after stimulation (typically after several minutes or less). Some embodiments include an additional polymer film (not shown) that separates the sweat stimulant gel 417 from skin 12 when the first molded polymer 412 is in the retracted position to help preserve the gel and prevent potential skin irritation. Once sweat is stimulated, the electroporation electrode 414 introduces electrical current into the skin 12 at low voltage (<5 V) and short (˜10 μs) pulses once every second or longer to increase the concentration of vasopressin that partitions into sweat from tissues surrounding the sweat gland.

In some embodiments, the electroporation electrode 414 can also function as a skin impedance sensor, which can provide information useful for controlling the electroporation or sweat stimulation functions. The wicking volume reducing component 410 transports stimulated sweat from the skin surface and carries the sweat sample to the sensing module sensors 422, 424, which would measure, e.g., Na⁺, K⁺, and pH. The sensing module may also include a sweat flow rate sensor. The wicking component 410 then transports the sweat sample to the specialized module sensors 432, which are EAB sensors for vasopressin. The vasopressin will be concentrated as water and small sweat solutes are transported through the forward osmosis membrane 437, into the osmosis material 436, and out of the sweat sample. Because the sweat sample will gradually increase in vasopressin concentration as the sample moves toward the pump 439, the sensors in the sensor suite 432 will see increasing amounts of vasopressin. By measuring vasopressin concentration with three sensors (each with ˜80× linear range), and with a measured sweat flow rate, the device determines the original sweat sample concentration of vasopressin. Finally, wicking pump 439, which could have a total wicking capacity of 10's to 100's of μL, absorbs the sweat sample, and at least partially pulls sweat sample flow through the device.

Embodiments of the present invention may be useful for a variety of sweat sensing applications. For example, low sweat rates enabled by embodiments of the present invention can also allow otherwise impractical sensing of some solutes. For example, a large sweat rate can cause sweat glands to generate significant quantities of lactate, making correlation between sweat lactate concentration and blood concentration impossible. Because embodiments of the disclosed invention are capable of detecting lactate at very low sweat generation rates, blood lactate that partitions into sweat can dominate over lactate generated by the sweat gland. Therefore, embodiments of the present invention enable improved sweat-based estimates of blood lactate. Embodiments of the present invention could also help in sensing of cytokines, which partition into sweat very slowly and require low sweat rates for accurate sweat concentrations that can be correlated with blood levels. Embodiments of the disclosed invention also improve other sensing applications by reducing the amount of stimulation needed for a given chronologically assured sampling interval by reducing the sweat volume needed by the sensors, which reduces needed sweat generation rate to refresh that sweat volume. Similarly, the present invention could also reduce the time for a new concentration of biomarkers to move from blood into sweat and onto the sensors, therefore providing sweat measurements that are closer to real time blood concentrations.

This has been a description of the disclosed invention along with a preferred method of practicing the invention, however the invention itself should only be defined by the appended claims.

What is claimed is:

1. A modular biofluid sensing device configured to be worn on a wearer's skin and capable of measuring at least one property of at least one analyte in the biofluid, comprising:
at least one primary module;
at least one sensing module; and
at least one specialized module.

2. The device of claim 1, wherein the primary module includes at least one of the following: a sweat stimulating component; a sweat rate sensor; a volume reducing component; a wicking volume reducing component; an electroporation component; and a skin impedance sensor.

3. The device of claim 1, wherein the sensing module includes at least one of the following component types: an ion selective electrode sensor; a biofluid flow rate sensor; a sweat rate sensor; a pH sensor; a temperature sensor; and a galvanic skin response sensor.

4. The device of claim 1, wherein the specialized module includes at least one of the following: an electrochemical aptamer-based sensor; an ion-selective electrode sensor; an amperometric sensor; a potentiometric sensor; an enzymatic sensor; an antibody sensor; an optical sensor; a surface-plasmon sensor; an acoustic sensor; a resonant sensor; a micro-electro-mechanical (MEMs) sensor; a biofluid sample concentration component; an osmotic pump component; a wicking component; and a sweat collection component.

5. The device of claim 1, wherein at least one module is disposable and at least one module is reusable.

6. The device of claim 1, wherein at least two of the modules are arranged in one of the following manners in relation to the wearer's skin: horizontally; vertically; and partially horizontally and partially vertically.

7. The device of claim 1, wherein at least two of the modules are integrated by one of the following: a fluidic contact; an adhesive; and a mechanical interaction.

8. The device of claim 1, including at least one of the following to facilitate fluidic, advective or diffusive contact among device components: a rigid molded polymer material; a rigid metal component; a self-leveling filler material; a hydrogel; and a memory foam material.

9. The device of claim 2, wherein the primary module includes a wicking hydrogel.

10. The device of claim 2, wherein the primary module includes a hydrophilic gold electroporation electrode.

11. The device of claim 2, wherein the primary module includes a skin impedance electrode sensor.

12. The device of claim 2, wherein the primary module includes a polymer wicking material with channel geometries to manage sweat sample flow.

13. The device of claim 2, wherein the primary module includes a sweat stimulation chemical and an iontophoresis electrode.

14. The device of claim 2, wherein the primary module includes sudo-motor axon reflex sweat stimulation.

15. The device of claim 2, wherein the primary module includes a sweat stimulation component having a first position relative to the wearer's skin and a second position relative to the wearer's skin, where the first position prevents sweat stimulation and the second position allows sweat stimulation.

16. The device of claim 3, wherein the sensing module includes the following: at least one sensor for Na⁺ or Cl⁻; at least one sensor for K⁺; at least one pH sensor; and at least one biofluid flow rate sensor.

17. The device of claim 3, wherein the sensing module includes at least one of a microfluidic component; and a wicking component.

18. The device of claim 4, wherein the specialized module includes at least one biofluid flow rate sensor.

19. The device of claim 4, wherein the specialized module includes at least one temperature sensor, and at least one pH sensor.

20. The device of claim 4, wherein the biofluid sample concentration component includes a forward osmosis membrane.

21. The device of claim 4, wherein the osmotic pump component includes one of the following: a wicking hydrogel; a suspension of a large organic salt; and a suspension of a sugar.
